(19) United States
(12) Patent Application Publication
(10) Pub. No.: US 2020/0155362 A1
(43) Pub. Date: May 21, 2020

(54) SELF-MONITORING COMPRESSION SUPPORTS

(71) Applicant: NEXTLIFE WEARABLES LIMITED, LONDON (GB)

(72) Inventors: Mark Pedley, Cheshire (GB); Adrian Ward, Barrow-Upon-Soar (GB)

(73) Assignee: NEXTLIFE WEARABLES LIMITED, LONDON (GB)

(21) Appl. No.: 16/628,282

(22) PCT Filed: Jul. 5, 2018
(86) PCT No.: PCT/GB2018/051913

§ 371 Date: Jan. 3, 2020

(30) Foreign Application Priority Data
Jul. 5, 2017 (GB) 1710807.7

(51) Int. Cl. A61F 13/08; A61B 5/00

(52) U.S. Cl. CPC A61F 13/08; A61B 2562/0247; A61B 2562/0271; A61B 5/6804

(57) ABSTRACT

The present invention relates to deformable supports that provide a compressive force on a human or animal limb or body and can self-determine that force and monitor it. The invention further relates to garments comprising those supports and an associated system for self-determination, monitoring and communicating the compressive force.

The present invention relates to deformable supports that provide a compressive force on a human or animal limb or body and can self-determine that force and monitor it. The invention further relates to garments comprising those supports and an associated system for self-determination, monitoring and communicating the compressive force.

### BACKGROUND

Utilisation of a compressive force on the whole or a part of the human or animal body is known to assist recovery from certain conditions, for example limb ulceration, where compression is part of a recommended healing plan.

Such garments are also useful in more general application, e.g. under, or as part of, sportswear to support the body during or after strenuous training.

Compression wear and/or garments typically comprise a single layer of material formed from strongly elasticated yarn to provide uniform compression when worn.

However, identifying whether a therapeutically beneficial level of compression is achieved, both on initial application of a support and after a period of use is difficult. Such a determination is often made subjectively, by either a user or clinician, and is open to interpretation.

Presently, there is no objective way to ascertain if the compression provided by a particular support is satisfactory and/or when increased compressive force might be beneficial which is an integral part of the support. Such a scenario may arise because there is not enough compressive force being applied for the size of the limb relative to the elastic strength/circumference of the support, or the requirement of force has changed due to a reduction in limb girth, for example due to healing or weight loss or sub-skin fluid movement, rendering the support functionally ineffective or, at least, sub-optimal or useless. Furthermore, being able to inherently determine fidelity of a compression support over time is highly desirable to ensure the life of the bandage is as expected. However, present bandages on the market do not provide such functionality.

There is no solution for accurately and easily determining the compressive force being applied to the limb by such a support or garment and it is in the context of this problem that the present invention has arisen.

### SUMMARY OF THE INVENTION

The present invention concerns a support configured to apply and self-determine compressive force (s) on a human or animal body or limb. The support comprises a deformable substrate having a first conductive pathway; a first sensor located in a first sensory zone of the support for measuring impedance resistance across the first conductive pathway; at least one pathway termination point for the sensor; and a detachable microprocessor or data collection interface operably connected with the pathway termination point for receiving resistance data from the sensor, wherein the detachable microprocessor converts the resistance data to pressure data via an algorithm, which is indicative of a first compressive force applied by the support in the first sensory zone.

The applicant has established that an arrangement in which the support itself can make an accurate determination of the mm Hg (millimetre of mercury) pressure at the sub-bandage interface between the support and the skin provides a useful solution to the problem set out above.

The support is able to determine and thereby indicate the pressure by actively measuring electrical resistance and/or electrical impedance which will change as a result of distortion or disruption to whole or part of the, at least one, conductive pathway(s) running along the support substrate. The support then converts the resistance measurements to determine, via an algorithm, a meaningful pressure reading at the sub-bandage interface in at least one sensory zone of the support.

Prior to the application a measurement is taken of the circumference or the limb for which the support is being applied and that information fed into the electronics via an app. Following the application of a support, the user or clinician can determine if there is sufficient compressive force being applied based on a first measurement of compressive force, which can be compared to therapeutically recommended pressures for a limb or body of a specified circumference, for example.

As a determination of compressive force can be made quickly and easily, quantitative monitoring becomes possible and can be used to enhance or optimise the healing or wound management process. Usefully, if force exertion on the limb has reduced over time due to a reduction in limb girth, this can be identified quickly and a new support selected to increase the support and maintain healing at an optimum rate. Failure to do this means that such a support would no longer promote healing through compression and thus would function sub-optimally or have no benefit at all.

Such a support is particularly useful because it measures the pressure using its own features and therefore more accurately determines the sub-bandage pressure than would be possible by external pressure measuring, whether carried out subjectively or by more technical means. Importantly, an accurate measurement is only possible because the support of the invention is undertaking real-time measuring of the mm Hg pressure at the sub-bandage interface between the support itself and either the skin or the pressure required on the protection layer that which covers the wound; such accuracy cannot be replicated in a remote or ambulatory situation by measurements taken externally to the support.

The sensor maybe a linear extension sensor provided along the first conductive pathway. In some embodiments the support further comprises a conductive pin at the termination point.

In some embodiments a conductive node is provided at each end of the first conductive pathway and the nodes function to activate and power the sensor, optionally where activation of the nodes is wireless.

In some examples the sensor is at least one linear extension sensor is integrally formed within the support. The support may comprise a plurality of linear extension sensors.

In some preferred embodiments the substrate of the support comprises a multiple conductive pathway corresponding to each linear extension sensor.

In some particularly preferred examples the substrate has two conductive pathways arranged perpendicular to one another; and the support comprises two independently operable sensors located in the first sensory zone for measuring impedance resistance independently across each of the respective perpendicular conductive pathways in the first sensory zone.

In addition, the support may further comprises a second independent sensor located in a second sensory zone and impedance resistance is measured across a second conductive pathway such that the detachable microprocessor converts that resistance data to pressure data which is indicative of a second compressive force applied in the second sensory zone.

The support may further comprises a third sensory zone, wherein a third independent sensor is located in the third sensory zone and impedance resistance is measured across a third conductive pathway such that the detachable microprocessor converts that resistance data to pressure data which is indicative of a third compressive force applied in the third sensory zone.

In some applications, for example in animals, the support may comprise further additional sensory zones to provide more than 3 sensory zones.

In preferred embodiments the first conductive pathway and corresponding sensor is provided by a linear electronic transducer with two termination points. Such linear electronic transducer typically further comprises a knitted or woven electro-conductive yarn.

The sensors may be programmable to undertake staggered activity in relation to the frequency and time of the sensing.

In the preferred embodiments above, the substrate having at least one conductive pathway(s) and/or at least one sensor(s) for measuring impedance resistance across the pathway can be in the form of a linear electronic transducer (LET), such as those developed by the present applicants.

Linear electronic transducers (LETs) previously described by the present applicants have been further developed and utilised to great effect within the present invention.

LETs are typically made from knitted or woven material being made, in part, from electro-conductive yarn. This may include silver or other metallic yarns. WO2009/093040 discloses an electro-conductive yarn within a knitted structure.

In this application the electro-conductive yarn of the LET forms part of a circuit configured to provide an indication of an electrical characteristic of the yarn and is used to obtain an indication of the deformation of the knitted structure. Stretch in knitted or woven yarn elongates closed loops reducing the number of contact points and changing the impedance of the transducer. In this way the LET inherently senses and convert the physical variation quantity into an electrical signal which, in the bandage of the present invention, is relayed via the termination point(s) to the microprocessor for conversion, determining the compressive force. The accuracy of the compressive force is improved by measurement of tension/extension at distinct positions or sensory zones, for example two or three, on the support (and thus respectively along the limb). Thus, ultimate calculation and determination of the compressive force becomes both locatable on the support bandage and cumulatively more accurate as a calculation in totem.

Importantly, the bandage of the invention recites defined independent sensory zones (of varying limb dimension) wherein the sensing function within each sensory zone remains autonomously performed and measured by a sensor. In preferred embodiments, the bandage comprises multiple independently measurable sensory zones. In further preferred embodiments there are two or up to three independently measurable sensory zones and hence forces are measured specifically to the limb at that location.

Thus, the improved sensing capability and zonal measurement determination, in conjunction with the other features of the bandage (including the elastic/deformable nature of the support in the associated area of the limb), enable the application and maintenance of an appropriately forced zonal compression pressure across the bandaged limb in a way that previous bandages have been unable to achieve.

Further, implementing LET within a single support or bandage permits independent sensing that provides new functionality. For example, the tension or extension sensed can be in a warp or weft direction of the fabric as described above. However, it may also permit sensing of both warp and weft, allowing extension and tension to be measured at the same time and converted independently.

In addition, the support of the invention permits changes in fidelity after a period of time during which extended wear, use, washing and processing has occurred and all of which may impact the lifespan of the bandage and thus indicate when the bandage needs replacing.

The microprocessor is preferably touched or affixed against the termination point. The termination point, which is preferably housed with a textile construction, consolidates the data of the LET and thus reports on the functionality of each sensor serially. More than one sensor terminal may be present in the arrangement and such terminals may be located at different positions in the bandage.

In non-LET specific embodiments the at least one sensor may be formed from printed circuitry and affixed to the substrate by a variety of means to meet at the termination point. In some embodiments at least one sensor, may be etched or printed onto the substrate to enable the substrate and the sensor to elongate equally and without tension or disruption existing between the materials.

In particular, where the sensor is integral to a yarn in the LET embodiment rather than applied onto a yarn or onto the support structure and the deformation of the change in the electrical characteristics of the yarn may be measured via the microprocessor or collected and conveyed wirelessly.

In the embodiment where the microprocessor collects inputs from the LET there may be two or up to three forces measuring longitudinal and latitudinal (flex) extension in addition to the application of surface force. Such an embodiment of the bandage therefore measures up to three forces (imposed upon it) rather than measuring the three forces applied to the substrate itself. The digital signal processing takes into account the variations of sensor dimensions, the specific area of the limb size which the specific sensor is reporting on, whilst determining the pressure being applied onto that specific area (as per the stage of medical direction and instruction) to collectively meet a pre-determined compression therapy requirement.

In some embodiments, the microprocessor may be activated by the user by means of a switch or by vibration or similar. It is important that the support has detachable electronics, including the microprocessor, since this enables the support to be easily cleaned for re-use or easily disposed of.

Measurement of the leg circumference can be fed into the algorithm at the outset, and before the application of the bandage, in order that the algorithm takes into account the extension of the sensor to arrive at a tension increase in the bandage substrate and thus equate that to a derived pressure as the bandage is applied.

As an LET is used (or since the sensor is integral to the substrate form the extension of the form is directly reflected in the extension of the sensor thus the form and the sensor report the same extension because the) resistance across (the sensor or LET) is proportional to its extension. From the resistance the tension is calculated.

An algorithm is used to convert tension to reflect a pressure, increasing and decreasing on a linear scale. Changes in pressure reported will result from, for example limb measurements changing. The algorithm will then monitor the stability of that applied measurement (pressure) and report changes to the applied pressure (from changes in bandage tension resulting from the sensor extension).

In the case of venous leg ulcers, changes in the affected area or zone will be reported throughout the duration of the applied form and coinciding with the treatment and progress or not of a wearer condition. Importantly the output relates to “sub-bandage” pressure and the force being applied (from the tension) will be correlated using a Pico press or similar measuring device which itself sits between the under layer of the bandage and the leg or leg shaping layer; the Pico press is an accepted and approved measurement device for sub-bandage pressure sensing.

Thus, once the support is in place, and the electronics connected, the microprocessor receives the resistance measurement from the LET or sensor(s) and determines a pressure measurement in the manner enabled by the invention.

The support may contain an integrated data capture/collection module which may be part of the support manufacturing process or may be integrated at a time subsequent to the support manufacture.

This collection device may connect wirelessly to the microprocessor which is placed in a different locale on the support as determined by the application and to offer the least amount of interruption to the application and use of the support or to reduce to tolerable levels the additionality of the electronics device for the purpose of comfort for the user.

The microprocessor records changes in the fidelity (stability) of the bandage during the period of its application. In some embodiments additional information relating to the bandage user's activity will be recorded and monitored (using an accelerometer or accelerometer gyro-meter) in order that a history of bandage changes can be created from the point the support is worn to its removal, or when it is changed in any way. Such records provide an important personal user history and may be converted into a report or log by a reporting module of the processor.

If there is change in the pressure (by a statistically significant value, which may pre-determined and stored as described above or be accessed by the microprocessor remotely) that change will be indicated.

In one embodiment pressure change(s) is indicated by a signal. Such a signal may be provided on the housing of the microprocessor itself, or the information triggering that signal communicated to a remote display (which receives that information from a communications module of the microprocessor). For example, the signal may be displayed on an APP of a phone so that the user or third party such as a nurse practitioner can easily read a visual signal or measurement remotely.

The microprocessor may therefore comprise a communication module to transmit or receive data, such as pressure measurements, remotely. A spasmodic and/or timed exchange between the support and a communication module may take place.

The data may be stored and/or collected and is subsequently transferred via one or more communication protocols to a receiver. Communications via a separate data receiver within range contact could contact the communication module of the microprocessor using a variety of protocols and platforms including but not limited to, Bluetooth protocol, RF (radio frequency), NFC (near field communications, NFMI (Near field magnetic induction), ZigBee or other protocol.

Data reports could be directed into another primary or secondary storage module to report alongside additional data input streams either synchronously or simultaneously or in a serial manner. For example, in one embodiment, the communications module may use a methodology to awake itself as a result of an external influence such as movement and/or when communication is required to operate in either a streaming or semi-streaming mode (or in order to collect the data stored and at intervals determined by the user (and subsequently disgorged to another platform compatible with the communication module).

The signal indicating a change may be a light-emitting diode (LED) or character display which provides information about whether the pressure is sufficient or a haptic vibration or other such alerting. Such a signal may also include a live reading of the measurement itself.

The support, in particular the microprocessor and/or its housing or a remote APP, may additionally comprise a variety of visual character displays to indicate the change in compressive force and/or the compressive force.

However, the indication that the force is insufficient may be a simple signal, for example one coloured LED to indicate sufficient pressure and another different coloured LED to indicate insufficient pressure. The signal may additionally or alternatively be an audible alarm or alert.

In some embodiments the indicator will include a number of adjacent LEDs, e.g. 1 to 8, corresponds to a pressure scale, whereby 1-3 adjacent LEDs are lit to indicate insufficient pressure, 4-6 indicate sufficient working pressure, whilst more than 6 signals a degree of compression that may not be tolerated well. Such a display may be particularly useful for active wound/injury management because it reduces the trial and error needed to identify and select an appropriate level of support. At the same time a wearers personal requirements can be taken into account to ensure the balance between optimal pressure application and user acceptability is achieved.

Such a support is advantageous as the user is immediately aware if changing to a new compressive support is required in order to maintain satisfactory compression. Equally interference of the support by the wearer is able to be monitored and recorded to inform third parties of changes in the supports status. In medical applications specifically, the healing process varies between patients and hence the change in level of compression required is unpredictable yet this is accounted for by the invention. The support of the invention therefore provides a personal healing management capability not seen in the art to date.

The microprocessor may comprise a power source. Power for the support and/or microprocessor could be kinetically generated from the activity of the wearer or collected using external sources such as light, heat, magnetic fields or use a battery or re-chargeable mode for its operation. The microprocessor may be housed within a pocket of the support, or may be attached to the conductive pin or may be otherwise sited within communication range elsewhere on the limb or the body and be secured therein temporarily during use of the support by the user. However, the microprocessor may also be detachable from the support and removed from the pocket as required.

In some embodiments, the electronics may be removed completely if the monitoring of substrate fidelity is not required.

Typically, the support is a provided as a length of wrapping material, such as a bandage. In such an embodiment at least one surface of the bandage support may additionally have an adhesive property configured to provide a temporary self-adhesive function. This ensures that the bandage does not become unwrapped from a bound position and maintains stability of the applied compression.

However, alternatively, the support may comprise an elastic material surround, such as a stocking or sleeve which can be pulled on or off a limb or part of the body. Such a surround may be seamless and may provide either uniform compression or tailored compression zones.

In some embodiments, the material of the support may be washable/waterproof to permit repeat usage.

In some embodiments, the support may be produced in a form factor which, rather being formed from than a continuous substrate such as a bandage, has a one piece construction with a number of straps as anchor points throughout the length of the support and whereby each support is measured independently using the LET and associated electronics.

In some embodiments it is envisaged that the support, including the substrate, may comprise further functionality for detecting other characteristic changes that may be helpful in optimising the use of the support.

In this regard, additional sensor validation may be incorporated within the support to report on changes during usage of the bandage to monitor additional limb attributes. An example may be a PPG optical sensor, a light sensor reporting diffraction, an RF sensor, a temperature sensor or another type of external sensor which maybe integrated, embedded, embroidered, knitted, woven or fused with the support of the invention.

Further embodiments may use the support in other ways e.g. using as a scaffold to monitor the presence of exudate/fluid, to monitor changes in blood pressure or pulse or temperature fluctuations or which reports colour change of appropriate yarns of the support to correspond with the extension, or which has another reportable outcome such as temperature and/or chemical sensing which may provide further information to aid wound/healing monitoring and management. In some embodiments it is envisaged that the support may comprise additional sensors for detecting other physical parameters such as an electronic tracker for movement, to monitor falls from rapid and un-anticipated body or posture state changes or distance and/or location of the support itself.

The invention extends to a garment comprising one or more supports as disclosed in the present applicant.

The invention also relates to a self-monitoring compression system that determines compressive force applied on a human or animal body or limb in at least one sensory zone of a compression support comprising: obtaining pressure data in a first sensory zone of the compression support having a first conductive pathway and a first sensor therein; measuring impedance resistance across the first conductive pathway; receiving and optionally storing resistance data via a microprocessor and thereafter converting resistance data to pressure data; comparing the pressure data to prescribed-set data to determine change in compressive force applied by the support, or comparing the pressure data over a pre-determined period of time to determine a statistically relevant change in pressure data indicative of a change in the compressive force; and communicating qualitative and/or quantitative information in relation to the compressive force to a receiver.

In embodiments the system determines and communicates qualitative and/or quantitative information in relation to compressive force in up to two further different sensory zones of the same support.

In further aspects, the invention provides a method for the treatment of an animal or human in need thereof comprising applying a support in accordance with any aspect or embodiment of the invention. Suitably the animal or human in need of treatment is one with an injury or condition where compressive force on the whole or a part of the human or animal body is known to assist recovery from certain conditions, for example limb ulceration. Suitably successful treatment may be a reduction of oedema, swelling, turgescence and/or inflammation.

It will be understood that although the support and systems of the present invention is predominantly applicable to a human or animal limb, in accordance with the examples described and illustrated herein, it may also be applicable to whole body.

### BRIEF DESCRIPTION

The present invention will now be further described with reference to the accompanying drawings in which:

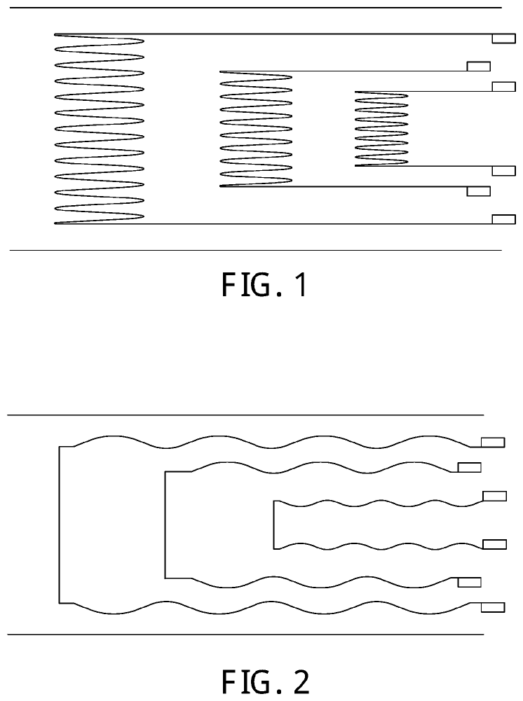

FIG. 1 shows a support according to the present invention and shows, as an example, 3 conductive pathways with termination points;

FIG. 2 shows a further example of a support according to the present invention and shows, as an example, conductive pathway sensor (integral);

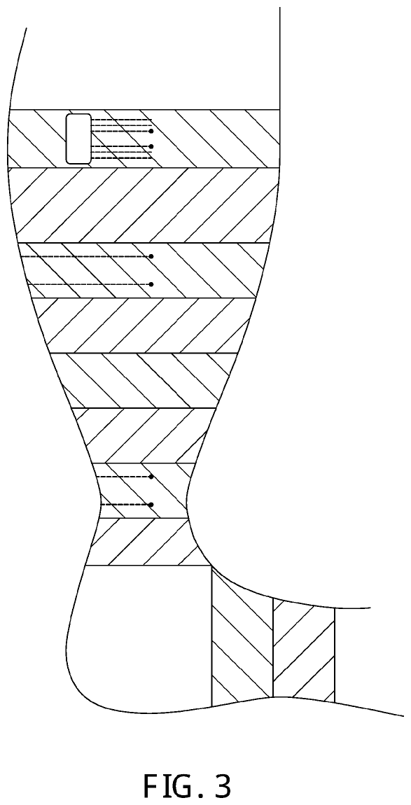

FIG. 3 shows an example of a support of the invention in use;

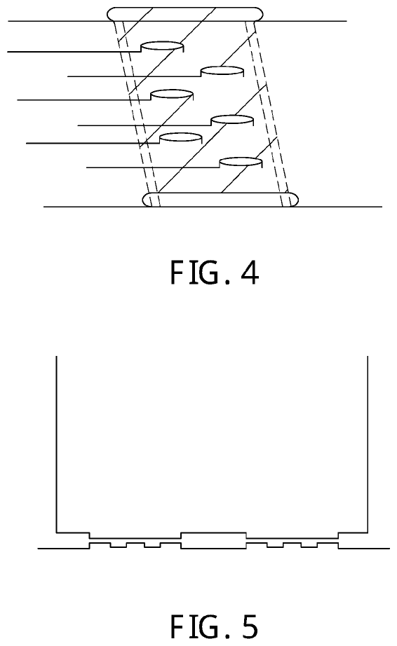

FIG. 4 shows yet a further example of the support of the invention with integral sensors; and

FIG. 5 shows the soft touch technology used at the termination points in some embodiments of the invention.

### DETAILED DESCRIPTION

FIG. 1 shows an embodiment of the support of the invention. The support comprises an inherently compression-based bandage or stocking that supports the limb or body. The substrate material applies a compressive force on a human or animal body or limb by virtue of its elasticity.

In other embodiments the substrate may be a non-compression based support. A bandage support may be wrapped multiple times around the supported limb and provided with an adjustable fastening or self-adhesive (cohesive) finish.

The substrate has 3 conductive pathways. There may be more or fewer conductive pathways provided on each support. In this example each of the three pathways has 2 termination points and impedance sensor associated therewith for measuring impedance resistance across the respective conductive pathway. The resistance across each pathway will change as a result of distortion, disruption to the whole or a part of the pathway by stretching or contraction of the material of the support. The sensor for each pathway detects the resistance and is read by electronics within a micro-processor (not shown) or by an integrated or integral electronics data collection device (not shown) which communicates with the micro-processor either of which are operably connected to the sensor. The applicant has developed knitted fabrics which are linear electronic transducers, which act as the support's substrate, inherently providing the features required such that resistance changes can be measured. The LET would provide the substrate, conductive pathways and the sensors needed with two termination points being required one for the supply and one for the return of each pathway. A micro-current is fed from the power source.

The fabrics used for such application include those described in WO2009/093040. For example, sensor deniers for this application can range between single ply 30/100 dtex (decitex) or up to 2 ply 100 dtex.

The nature of the electronic connection by the termination point(s) to the electronics of the microprocessor may be via a conductive pin, popper or press stud, magnetic or other type of mechanical method. However, the applicant's associated soft contact sensor technology in which the fabric of the support is seamlessly interconnected with the electronics of a microprocessor or a collection device which is integrated into the support/bandage could also be used and is described further below.

Alternatively, the electronics interface conjoining the termination points of the pathways could be constructed from a flexible material upon which electronics are sited and which is embedded into the support ether at the time of manufacture of attached, in a permanent manner, at the time of application.

Furthermore, as an alternative, the collection device could be connected using wireless frequencies to either the micro-processor which is sited within connectivity range of the support and the sensors and is separately attached or carried onto or by a limb or is connected using radio frequency technology to a hand-held or similar type reader device. The microprocessor receives the signal from the sensors and converts the resistance signals into a pressure measurement for each.

Here, the conductive pathway may be provided by the linear electronic transducer, integrally formed within a support either during manufacture or subsequently as described above.

In some embodiments the invention may therefore comprise a secondary or alternate sensor. The sensor may be using a flexible or fluid sensory system integrated within the support and which measure pressure from deformity of the system whilst measure surface pressure force, thus measuring longitude, latitude and vertical forces simultaneously the communication of the data being deployed using similar methodologies as described above.

In another embodiment the sensor may be applied onto the support material using a variety of affixing methods such as etching, printing, bonding, fusing, gluing etc. to adopt the characteristic elongated attributes of the support material to measure the deformation of the sensor as part of the material via a collected signal.

The fabric substrate comprises one or more row, or course, of extensible woven or knitted fabric, each row comprising stitches. The substrate or support may include a cotton property. Each stitch comprises loops which interconnect with the adjacent stitches within the same row and also the adjacent stitches in the previous and subsequent rows, i.e. those stitches above and below. As a result of stitch interconnectivity, if a force is applied across the fabric, each loop deforms from a substantially circular configuration into an elongate oval configuration enabling the fabric as a whole to stretch in the direction in which the force is applied. Because this deformation arises as a result of the knitted fabric structure, it is apparent even if the yarn itself is inextensible.

In this example, several rows of the knitted support can be provided in the form of electro-conductive yarn, thereby forming one or more separate conductive pathways. Each of the one or more extensible sensors has two pathways for carrying the electrical displacement signals. In the case of a plurality of sensors, each one is separate to each-other and reacts in a sequential way autonomously as the support is applied along the length of a limb or body. When the support is in an un-extended condition, each adjacent stitch is configured as a substantially closed loop. When the support is stretched, each loop elongates and the number of contact points is reduced thus changing the impedance of the conductive pathway.

In some embodiments, the knitted fabric may be formed from a low modulus yarn that is typically elastomeric. In that configuration, the fabric can deform further than an equivalent knitted structure formed from an inextensible yarn, because the yarn itself can deform under an applied force. The conductive yarn pathway is threaded through this substrate, and is not elastic but has pockets of longer length. As the adjacent elastic yarn is stretched the conductive yarn extends from a wound configuration to an open configuration. The resistance across the conductive pathway is therefore changed as the fabric is stretched in the direction of the elongated conductive yarn.

In order to optimise the data received by the microprocessor sensor, the transduction along the pathway in the LET embodiments can be enhanced by providing 2 or more conductive yarns immediately adjacent one another effectively forming the same conductive pathway. Data from the 2 conductive yarns received by the sensors can be averaged to provide a single resistance data value. Alternatively, this arrangement can provide a built-in functioning check. In such a case, the 2 separate resistance measurements can be compared to check that they do not differ by more than a predetermined threshold amount. If they do differ by more than a predetermined amount, this could indicate that at least one of the strands has been damaged and the data received from it should therefore no longer be provided for conversion to pressure data.

As one skilled in the art will appreciate other variations of electronic transducers may be utilised. In a simple embodiment, a LET with a single conductive pathway may be provided by a conductive yarn and thus only one sensing system is required to be connected to the microprocessor or to a data collection termination point. Such embodiments may be useful with short supports e.g. useful for children, or where the limb or area of body to be supported is very short or limited in length e.g. wrist.

In FIG. 1 specifically, a zig-zag extends and with a number of peaks separating lengthwise and it creates a displacement of signal going into the return track of the sensor. The tracks are functional in that one track from the termination point to the sensor carries a supply flow of electricity and as the electricity flows through the sensor it's resistance is stabilised and the returning track records the power. When the sensor is stretched it is stretched length ways (left to right) not widthways (top to bottom on the drawing. This causes a resistance change in the sensor (as the power becomes interrupted by the separation of the loops) and it is that change which is measured.

FIG. 2 shows the same multi-sensor array integral within a support but not requiring the plurality of sensor configuration which creates a zig-zag in FIG. 1. A multi-sensor support arrangement applied to a limb with 3 conductive pathways, this time fixed within a specific zone of the support that requires measurement of resistance. Such pathways, are each extendable within their zone only as the support is applied. Each pathway comprises at least 1 sensor to measure the resistance across the fixed length of the conductive pathway in that particular compression zone and this can be pre-programmed if required into the sensor electronics via the app and then be translated, via the algorithm, to provide a reading of the average pressure exerted across the conductive pathway. Such a pressure measurement may provide data in relation to the specific part of the limb measured, e.g. one may relate to the ankle, whereas another may relate to the shin/calf and a further may relate to under the knee.

The conduction pathways are knitted with additional conductive yarn which can be held within the support by the elasticity of the support material. When the support is extended (stretched) the additional thread straightens and the resistance created by the loops when flaccid is reduced and the changes are measured as linear extension change and converted by the algorithm to a pressure formulae. When it is in its resting state the electricity flows with enforcement, when it is extended it changes and flows more freely.

FIG. 3

In this embodiment the support is wrapped around the limb with a successive overlap. The application of the support is such that the overlap, of each successive length being wrapped upon the preceding, one results in between 33%>50% of the width of the preceding layer subsequently occurring layer upon layer. There is a 50% overlap (if half of the preceding layer is covered). This embodiment creates and applies stability and reinforces the pressure applied by the support. It is possible that the amount of overlap required from 50% to 25/33% (thus shortening the overall length requirement of the support and reduce subsequent manufacturing costs) due to the accuracy of the applied bandage and by moderating the amount of elasticity within the support to provide and maintain the overall compression required of the support as the stretch of the support is applying the required pressure to the limb at the right position.

FIG. 4

In this embodiment, the support is shown with integral sensors which may be made during manufacture. The sensors converge at a termination point, housed in a pocket, or it may be a flexible termination point. The connection is via either mechanical or soft touch, as described throughout this disclosure. The data collection technology may include the multi-processor or it may provide a location point for another rigid or flexible interface. In some embodiments the interface may collect the sensor data and store it until the electronics (or a separately introduced set reader) collects the data for extrapolation, processing, reading and/or forwarding on to another device. However, in some embodiments the data would be processed as it is collected, communicated onto another device and may not be stored in its raw form.

FIG. 4 illustrates the embodiment where one or more pathway termination points (such as the 6 shown here) are comprised within a housing which could be a pocket (that is additional to or formed as part of the support or held by a retaining method which secures an interface for whatever period of time is required). The array of particular pathways as shown is not necessarily limited in number, design or layout and other embodiments are envisaged within the scope of the present application.

FIG. 5

In this embodiment the sensor termination points and the opposing interface have raised profiles which, when applied together, ensure that a reliable and constant contact is made and without the requirement of mechanical components. The applicant has shown that repeatable results in data transference can be generated using this embodiment and this data can then be subsequently analysed.

The force applied to the outer surface or top of the interface is applied by either a pocket covering which has been extended from its resting state and when operational under pressure due to the pocket accommodating the inclusion of the electronics or interface mechanism.

Alternatively there may be integral location points set within or added subsequently to the support the purpose of which locate the interface into its correct position and force said interface in contact with the sensors terminations points. In another form, mechanical poppers, magnets or snap connectors or other such devices may be adhered onto the termination points to ensure that a solid contact is made and the interface is then snapped into place on the reverse side of the connectors e.g. a female connector on the interface vs. a male connector on the textiles termination points.

In another embodiment it is possible to utilise an optical connector which has one part embedded within the interface to provide an infra-red or similar signal with the other opposing side having a reflector/reader which aligns the interface and facilitates the transfer of data.

There are a number of combinations and variations of each of the essential features (and non-essential or additional components) which forms different embodiments and these will be clear to the skilled person as being within the scope of the present invention. Such features may include some or all of the following components:

Sensors and Staggering of Signals

Each pathway may be provided with an impedance sensor or one sensor can serve multiple pathways via staggering.

The sensors can be configured to sense the impedance at regular intervals, for example at intervals of a fraction of a minute or 1 minute, 5 minutes, 10 minutes, 20 minutes, 30 minutes or hourly. For example, if a support is configured with five conductive pathways, pathways A, B, C, D, and E each sensor may be configured to provide a reading every five minutes, but staggered at one minute intervals so that a reading is taken every minute from one of the sensors.

Furthermore, the sensors can be configured to the requirements of a wearer to provide the best compression to optimally treat the patient wearer's current condition which, in the case of a leg ulcer, changes from application to application and from time period to time period. This may result in a bespoke solution tailored for generic treatment routines or individualised for patient treatment plans. Input into the electronics which record the variances within the sensors is made via a compatible and is programmable by a piece of electronics or software, such as a smart phone or tablet computer which can be operably connected thereto.

Furthermore, the sensors can be configured to provide additional readings in response to a change in reading in one sensor. In the above example, if the impedance across pathway C changes by more than a predetermined threshold amount, then additional data may immediately be requested for the impedance across pathways D, E, A and B. These data may be obtained sequentially at a minimum temporal spacing, for example at one second intervals.

Termination Points

At least one pathway termination point is required at the end of the one or more conductive pathways in all examples shown herein. Where a number of conductive pathways are provided within a single support, all of the conductive pathways may otherwise be grouped for ease of connection or brought together in a single termination point.

Convenient communication of resistance data from the conductive pathway/sensor may be provided via a conductive stud or pin to the microprocessor.

Additionally, a rigid or flexible interface may be applied to or formed as part of the support and may contain a data collection facility or which may solely collect and communicate the data either dynamically or after a pre-prescribed period of time or which is in response to a stimulate appeal upon the interface by a configured device which is made to collect the data.

However, other means of connecting to electronics of the microprocessor are envisaged, including but not limited to the applicant's own developments in fabric-electronic interfacing technology as described in WO2014/188171. In such a case an improved contact interface between the electronics of the microprocessor and termination point of the conductive pathway is formed. For example, a knitted conductive pathway as described previously may terminate at a point within the substrate where a fabric pocket for housing the electronics of the microprocessor is formed. An additional base layer of fabric thickness within the pocket projects the surface of the electrical connectors of the microprocessor, when housed within the pocket, toward the conductive pathway surfaces.

Further, the electrical connectors of the microprocessor may include shims so they themselves are urged towards the desired points of contact at the termination points in the fabric.

Microprocessor or Data Collection Interface

A detachable microprocessor is operably connected with the at least one pathway termination point for receiving impedance resistance data. Data obtained by the sensor or sensors provides a reading of the resistance across at least part of the support and may be received, stored and processed in the same component within the device. The functionality of the microprocessor may range from that of a simple switching technique, without the need for integrated micro-processing to an advanced type of data collection which includes the ability to collect, process and communicate one or more signals concurrently to a receiving device. This latter, more detailed, functionality is described below.

As an alternative to a microprocessor, a detachable or non-detachable interface constructed of flexible or semi-rigid substrate may comprise minimal electronic componentry. In some embodiments, the power requirement for such electrics is minimal and maybe derived from the haptic or kinetic movement of the individual sufficient to allow the data to be carried into and stored upon the interface. The data may be stored in the interface before the interface then engages with another device to download and/or respond to further operational instructions. As such, it is envisaged that data collection, as compared to data processing, may be undertaken by separate parts of the device in some embodiments of the invention.

Algorithm

The microprocessor includes one or more algorithms for converting the impendence resistance data to pressure data to determine the compressive force across one or more parts of the support of the invention.

The algorithm utilises the measured resistance and the radius of the cylindrical limb or body with Laplace's Law to determine the pressure applied along the conductive pathway.

Where multiple sensors are provided along a conductive pathway, each sensor measures the resistance across a part of the length of the conductive pathway and each reading can be translated, via the algorithm, to provide a pressure reading for that part of the pathway. These individual readings can then be amalgamated to provide a pressure profile for the full length of the conductive pathway.

Housing

In some preferred embodiments there is provided a housing, which accommodates the microprocessor, the power supply and the electronics which perform the algorithm and also a memory in which data can be stored pertaining to the form factor calculated by the algorithm. The memory is configured to store data output from the algorithm and also to store data pertaining to acceptable ranges of pressure applied by the support. The memory provides for a learning capability derived from the data recorded. Therefore, a mean or steady state reading can be retained from a previous use in order to set a correct fitting of the support for a subsequent use. The housing may be formed from plastic or polymer and if conductive connect shims are chosen these may be formed in an integral manner when the housing is moulded and machined or they may be attached as a separate exercise once the housing is formed and the electronics installed. The housing may be encapsulated or sealed to offer a water resistant solution.

The housing may include the conductive pathway termination point in the form of a conductive pin, stud or soft touch contact membrane which provides an interface between the conductive pathway within the support and the microprocessor in which the algorithm is run.

The electronics housing may be a tailored touch enabled electronic housing which may additionally be configured to communicate data external to the electronics itself. This communication may be achieved via a range of wireless protocols, e.g. Wi-Fi, RFID, NFC, the internet and/or mobile telecommunications systems, enabling data to be retrieved and monitored remotely by a third party. There may be a LED or similar display on the housing and programmable instruction buttons may also be applied.

The software running a microprocessor would be updatable via over-the-air updating, it would use wireless technologies to perform self-checking and allow for external instructions to be fed into the electronics in order that greater functionality and/or accuracy and/or flexibility is afforded to the use of the support.

Display

In one embodiment the housing comprises a display configured to show a visual representation of the output of the algorithm. This may be a numerical display, showing the numerical value of the pressure applied by the support and/or in one or more distinct zones or areas across the support.

Alternatively, it may be a simple visual representation of the status of the support. For example, a green light if the pressure lies within acceptable limits, a red light if the pressure exceeds a predetermined upper limit for the acceptable pressure and a blue light if the pressure falls below a predetermined lower limit for the acceptable pressure.

In some embodiments, the display may be audible and comprise an alarm which initiates an alert when the pressure across the conductive pathway falls outside of the acceptable pressure range set and stored within the memory. The alarm may be configured to repeat whenever the pressure remains outside the acceptable range for more than a predetermined time, which may be a few seconds, a few minutes or an hour or more.

In another embodiment the displays mentioned above may be located on a mobile devices such as a smart phone, hand help tablet or similar and the data collected may be communicated as raw data transference, pre-processed data transference or full secure data transference whichever is required by the application. Fully developed applications for use with this technology are useable on a mobile device, such as a smart phone such that the operator, user or third party can remotely receive information visually and/or receive audible alarms to indicate when change occurs.

1. A support configured to apply and self-determine compressive force (s) on a human or animal body or limb, the support comprising:
a deformable substrate having a first conductive pathway;
at least one sensor located in a first sensory zone of the support for measuring impedance resistance across the first conductive pathway;
at least one pathway termination point for the sensor; and
a detachable microprocessor operably connected with the pathway termination point for receiving resistance data from the sensor,
wherein the detachable microprocessor converts the resistance data to pressure data, via an algorithm, which is indicative of a first compressive force applied by the support in the first sensory zone.

2. The support of claim 1, wherein the sensor is a linear extension sensor provided along the first conductive pathway.

3. The support of claim 1, wherein the support further comprises a conductive pin at the termination point.

4. The support of claim 1, wherein a conductive node is provided at each end of the first conductive pathway.

5. The support of claim 4, wherein the nodes function to activate and power the sensor, optionally where activation of the nodes is wireless.

6. The support of claim 1, wherein the sensor is a linear extension sensor integrally formed within the support.

7. The support of claim 1, wherein the support comprises a plurality of linear extension sensors.

8. The support of claim 7, wherein the substrate comprises a conductive pathway corresponding to each linear extension sensor.

9. The support of claim 8, wherein the substrate has two conductive pathways arranged perpendicular to one another; and the support comprises two independently operable sensors located in the first sensory zone for measuring impedance resistance independently across each of the respective perpendicular conductive pathways in the first sensory zone.

10. The support of claim 8, wherein the support comprises a second independent sensor located in a second sensory zone and impedance resistance is measured across a second conductive pathway such that the detachable microprocessor converts that resistance data to pressure data which is indicative of a second compressive force applied in the second sensory zone.

11. The support of claim 10, wherein the support comprises a third sensory zone, wherein a third independent sensor is located in the third sensory zone and impedance resistance is measured across a third conductive pathway such that the detachable microprocessor converts that resistance data to pressure data which is indicative of a third compressive force applied in the third sensory zone.

12. The support of claim 1, wherein each conductive pathway and corresponding sensor is provided by a linear electronic transducer with two termination points.

13. The support of claim 12, wherein the linear electronic transducer comprises a knitted or woven electro-conductive yarn.

14. The support of claim 1, wherein the support further comprises an indicator to communicate compressive force (s) to the user.

15. The support of claim 1, wherein the microprocessor comprises a communication module configured to transmit or receive data remotely.

16. The support of claim 1, wherein the microprocessor has a protective seal for sterilisation.

17. The support of claim 1, wherein the support is an elasticated wrap, bandage, surround, stocking or sleeve.

18. The support of claim 1, wherein the support further comprises at least one surface having an adhesive property to provide temporary self-adhesion.

19. (canceled)

20. The support of claim 1, wherein the support further comprises temperature and/or chemical sensors.

21-23. (canceled)

24. A self-monitoring compression system configured to determine compressive force applied on a human or animal body or limb in at least one sensory zone of a compression support, the self-monitoring compression system being configured to carry out a method, the method comprising:
obtaining pressure data in a first sensory zone of the compression support having a first conductive pathway and first sensor therein;
measuring impedance resistance across the first conductive pathway;
receiving and optionally storing resistance data via a microprocessor and thereafter converting resistance data to pressure data;
comparing the pressure data to prescribed data to determine change in compressive force applied by the support, or comparing the pressure data over a pre-determined period of time to determine a statistically relevant change in pressure data indicative of a change in the compressive force; and
communicating qualitative and/or quantitative information in relation to the compressive force to a receiver.

25-27. (canceled)
